(19) United States
(12) Patent Application Publication
(10) Pub. No.: US 2013/0096654 A1
(43) Pub. Date: Apr. 18, 2013

(54) Vestibular Implant System with Low Battery Alert

(71) Applicants: Andreas Jäger, Reith bei Seefeld (AT); Carolyn Garnham, Matlock Derbyshire (GB); Roland Hessler, Innsbruck (AT); Martin Zimmerling, Patsch (AT); Charles Coleman Della Santina, Towson, MD (US); Gene Fridman, Santa Clarita, CA (US)

(72) Inventors: Andreas Jäger, Reith bei Seefeld (AT); Carolyn Garnham, Matlock Derbyshire (GB); Roland Hessler, Innsbruck (AT); Martin Zimmerling, Patsch (AT); Charles Coleman Della Santina, Towson, MD (US); Gene Fridman, Santa Clarita, CA (US)

(73) Assignee: MED-EL ELEKTROMEDIZINISCHE GERAETE GMBH, Innsbruck (AT)

(21) Appl. No.: 13/526,724

(22) Filed: Jun. 19, 2012

Related U.S. Application Data
(60) Provisional application No. 61/498,760, filed on Jun. 20, 2011.

(51) Int. Cl. A61N 1/36

(52) U.S. Cl. CPC A61N 1/36142
USPC 607/63

(57) ABSTRACT

A vestibular implant system is described which includes an implantable vestibular stimulator providing a vestibular stimulation signal to electrically stimulate target neural tissue for vestibular sensation by a patient. A patient warning alarm process alters the stimulation signal when a given alarm condition occurs to change the vestibular sensation of the patient thereby warning the patient of the alarm condition.

This application claims priority from U.S. Provisional Patent Application 61/498,760, filed Jun. 20, 2011, which is incorporated herein by reference.

### TECHNICAL FIELD

The present invention relates to implantable stimulation systems, and more specifically to a vestibular implant system with a low battery alert functionality.

### BACKGROUND ART

A normal ear directs sounds as shown in FIG. 1 from the outer ear pinna 101 through the generally cylindrical ear canal 110 to vibrate the tympanic membrane 102 (eardrum). The tympanic membrane 102 moves the bones of the middle ear 103 (malleus, incus, and stapes) that vibrate the cochlea 104, which in turn functions as a transducer to generate electric pulses to the brain that are interpreted as sounds.

In addition, the inner ear also includes a balance sensing vestibular system which involves the vestibular labyrinth, its three interconnected and mutually orthogonal semi-circular canals: the superior canal 106, posterior canal 107, and horizontal canal 108 (as well as the otolith organs 116 in the utricle and saccule of the inner ear. The canals and otoliths of the vestibular labyrinth contain hair cells 118 in a viscous endolymph 117 to sense head orientation and head movements, thereby activating vestibular nerve fibers 119 that send an electrical balance signal to the brain 105.

In some people, the vestibular system is damaged or impaired. Such vestibular dysfunction can cause balance problems such as unsteadiness, vertigo and unsteady vision. This can be a significant handicap in everyday life. To treat such problems, Electrical stimulation of the vestibular system can help to restore the balancing function, and vestibular implants are currently under development to provide such an artificial balance signal.

FIG. 1 also shows some components of a vestibular implant system such as is described in U.S. Patent Application 61/366,345 (incorporated herein by reference). An external movement signal (from one or more sensors not shown) is processed by an external processor 111 to produce a vestibular stimulation signal. An external transmitter coil 112 couples the stimulation signal through the skin to an implanted receiver coil 113. Implanted vestibular stimulator 114 than delivers the stimulation signal through an electrode lead 109 to vestibular stimulator electrodes 115 that electrically stimulate target neural tissue such as the semicircular canals 106, 107, 108, one or both otolith organs, and/or the vestibular nerve 105 or ganglion for vestibular sensation by the patient as a balance signal.

One of the challenges for vestibular implant systems is the relatively significant amount of power required by the system. Gyro-based movement sensors have relatively high power consumption, and this requires a relatively large battery either in the implanted part or in an external part of the vestibular implant system and/or relatively frequent battery re-charging cycles. Both the onset of electrical stimulation when the vestibular implant is being activated and the ending of electrical stimulation when the system switches off (e.g. when the battery is depleted) will be required from time to time. Both are challenging situations since the respective changes in electrical stimulation patterns can result in severe patient discomfort (e.g., vertigo). Certain situations, such as changes in the electrical stimulation patterns, can also be potentially dangerous; for example, when electrical stimulation of the vestibular organ suddenly stops while the patient is driving a car, especially when this occurs unexpectedly. Thus it is important for the patient to be alerted well in advance before the battery stops functioning and electrical stimulation of the vestibular system is switched off

### SUMMARY

Embodiments of the present invention are directed to a vestibular implant system and corresponding method of operating such a system. An implantable vestibular stimulator provides a vestibular stimulation signal to electrically stimulate target neural tissue for vestibular sensation by a patient. A patient warning alarm process alters the stimulation signal when a given alarm condition occurs to change the vestibular sensation of the patient thereby warning the patient of the alarm condition.

In further specific embodiments, the alarm condition may presage a change in operating functionality of the system whereby the altered stimulation signal warns the patient of the change. For example, the change in operating functionality may include a reduction in operating functionality of the system and/or a termination of operating functionality of the system.

The alarm condition may include a low battery condition, and/or a reduced or lost connection with an external portion of the system. There may be multiple different alarm conditions such that the alarm process alters the stimulation signal in a different distinctive way for each different alarm condition. The alarm process may alter the stimulation signal to a constant pacing mode instead of depending on sensory inputs. In addition or alternatively, the alarm process may alter the stimulation signal for a brief period to warn the patient of the alarm condition and then shift back to allow an unaltered stimulation signal. In such an arrangement, it may be useful to repeat the brief period of altering multiple times. And the alarm process may terminate the stimulation signal during the brief period of altering.

Embodiments of the present invention also are directed to another vestibular implant system and corresponding method of operating such a system. An implantable vestibular stimulator provides a vestibular stimulation signal to electrically stimulate target neural tissue for vestibular sensation by a patient. A patient warning alarm process activating a patient alarm signal on a separate sensory channel independent of the stimulation signal when a given alarm condition occurs to warn the patient of the alarm condition.

In further such embodiments, the alarm condition may presage a change in operating functionality of the system whereby the alarm signal warns the patient of the change. For example, the change in operating functionality may include a reduction in operating functionality of the system or a termination of operating functionality of the system. The alarm condition may include a low battery condition, or a reduced or lost connection with an external portion of the system. There may be multiple different alarm conditions with a different distinctive patient alarm signal for each alarm condition. The patient alarm signal may be routed via other sensory systems of the patient than the vestibular organ. The alarm signal can be a vibratory signal, an auditory signal, a visual signal or an electrical stimulation signal. Alarm signals can be elicited by an externally worn unit of the vestibular implant system and/or by its implantable unit.

### BRIEF DESCRIPTION OF THE DRAWINGS

FIG. 1 shows various anatomical structures in a human ear including one specific embodiment of a vestibular implant system.

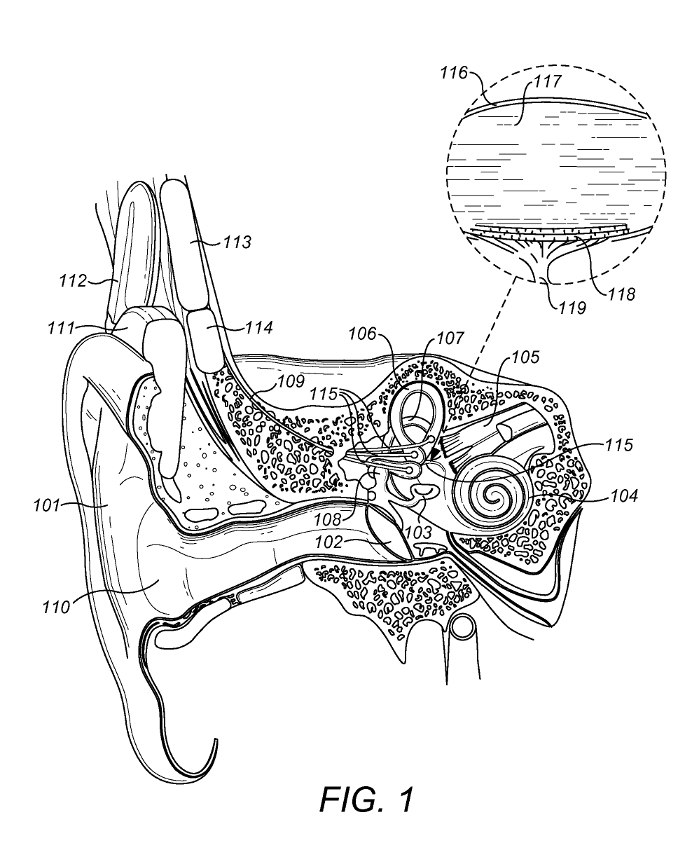

### DETAILED DESCRIPTION

Embodiments of the present invention are directed to a patient warning alarm for a vestibular implant system warning the patient of one or more alarm conditions such as low battery power. Various specific mechanisms are available, some of which are private to the patient and others of which are public and can intentionally also be recognized by persons in the vicinity of the patient.

For example, a private patient warning arrangement can be implemented in an implantable vestibular stimulator 114 that provides a vestibular stimulation signal to electrically stimulate target neural tissue of semi-circular canals (106, 107, 108) and/or otolith organs 116 for vestibular sensation by a patient. A patient warning alarm process (e.g., a software process running within the vestibular stimulator 114) then alters the stimulation signal when a given alarm condition occurs to change the vestibular sensation of the patient thereby warning the patient of the alarm condition.

For example, the alarm condition may include a low battery condition, and/or a reduced or lost connection with an external portion of the system. Another alarm condition may presage a change in operating functionality of the system such as a reduction in operating functionality of the system and/or a termination of operating functionality of the system whereby the altered stimulation signal warns the patient of the change. Thus, in some embodiments there may be multiple different alarm conditions such that the alarm process alters the stimulation signal in a different distinctive way for each different alarm condition.

The alarm process may alter the stimulation signal to a constant pacing mode of the vestibular stimulator 114 independent of sensory inputs. In addition or alternatively, the alarm process may alter the stimulation signal from the vestibular stimulator 114 for a brief period to warn the patient of the alarm condition and then shift back to allow an unaltered stimulation signal. In such an arrangement, it may be useful to repeat the brief period of altering multiple times. And the alarm process may terminate the stimulation signal from the vestibular stimulator 114 during the brief period of altering.

A private patient warning arrangement can be implemented in an implantable vestibular stimulator 114 that provides a vestibular stimulation signal to electrically stimulate target neural tissue of semi-circular canals (106, 107, 108) or otolith organs 116 for vestibular sensation by a patient. A patient warning alarm process (e.g., a software process running within the vestibular stimulator 114) then activates a patient alarm signal on a separate sensory channel independent of the stimulation signal when a given alarm condition occurs to warn the patient of the alarm condition. For example, the alarm condition may include a low battery condition, and/or a reduced or lost connection with an external portion of the system. Another alarm condition may presage a change in operating functionality of the system such as a reduction in operating functionality of the system and/or a termination of operating functionality of the system whereby the altered stimulation signal warns the patient of the change. Thus, in some embodiments there may be multiple different alarm conditions such that the alarm process alters the stimulation signal in a different distinctive way for each different alarm condition. The patient alarm signal may be a vibratory signal, an auditory signal, a visual signal or an electrical stimulation signal.

In the foregoing, references to vestibular implant systems should be understood broadly to include all implantable arrangements that provide stimulation signals affecting the balance sensing system. Specifically such arrangements may or may not include motion sensors, whether internal or external. For example, a vestibular implant system without motion sensing signals may be useful for treatment related to Meniere's disease and may be thought of as a Meniere's implant. And vestibular implant arrangements may also be integrated together with other related implantable systems such as middle ear implants, cochlear implants, bone conduction implants, auditory brainstem implants, etc. And the stimulation signals may occur either by electrical means as commonly used in current cochlear implant technology, by optical means (e.g. as disclosed in U.S. Pat. No. 7,488,341 and in U.S. patent application Ser. No. 12/368,548; both incorporated herein by reference), by mechanical means (e.g. as disclosed in U.S. patent application Ser. No. 11/193,034, incorporated herein by reference), and or some combination of different types of stimulation signals. And specific alarm signals and alarm conditions in any of the described variations can be combined.

Embodiments of the invention may be implemented in part in any conventional computer programming language. For example, preferred embodiments may be implemented in a procedural programming language (e.g., “C”) or an object oriented programming language (e.g., “C++”, Python). Alternative embodiments of the invention may be implemented as pre-programmed hardware elements, other related components, or as a combination of hardware and software components.

Embodiments can be implemented in part as a computer program product for use with a computer system. Such implementation may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web).

Although various exemplary embodiments of the invention have been disclosed, it should be apparent to those skilled in the art that various changes and modifications can be made which will achieve some of the advantages of the invention without departing from the true scope of the invention.

What is claimed is:

1. A vestibular implant system comprising:
an implantable vestibular stimulator for providing a vestibular stimulation signal to electrically stimulate target neural tissue for vestibular sensation by a patient; and
a patient warning alarm process for altering the stimulation signal when a given alarm condition occurs to change the vestibular sensation of the patient thereby warning the patient of the alarm condition.

2. A system according to claim 1, wherein the alarm condition presages a change in operating functionality of the system whereby the altered stimulation signal warns the patient of the change.

3. A system according to claim 2, wherein the change in operating functionality includes a reduction in operating functionality of the system.

4. A system according to claim 2, wherein the change in operating functionality includes a termination of operating functionality of the system.

5. A system according to claim 1, wherein the alarm condition includes a low battery condition.

6. A system according to claim 1, wherein the alarm condition includes a reduced connection with an external portion of the system.

7. A system according to claim 1, wherein the alarm condition includes loss of connection with an external portion of the system.

8. A system according to claim 1, wherein the given alarm condition includes a plurality of different alarm conditions, and wherein the alarm process alters the stimulation signal in a different distinctive way for each alarm condition.

9. A system according to claim 1, wherein the alarm process alters the stimulation signal to a constant pacing mode independent of sensory inputs.

10. A system according to claim 1, wherein the alarm process alters the stimulation signal for a brief period to warn the patient of the alarm condition and then shifts back to allow an unaltered stimulation signal.

11. A system according to claim 10, wherein the alarm process repeats the brief period of altering a plurality of times.

12. A system according to claim 10, wherein the alarm process terminates the stimulation signal during the brief period of altering.

13. A vestibular implant system comprising:
an implantable vestibular stimulator for providing a vestibular stimulation signal to electrically stimulate target neural tissue for vestibular sensation by a patient; and
a patient warning alarm process for activating a patient alarm signal on a separate sensory channel independent of the stimulation signal when a given alarm condition occurs to warn the patient of the alarm condition.

14. A system according to claim 13, wherein the alarm condition presages a change in operating functionality of the system whereby the alarm signal warns the patient of the change.

15. A system according to claim 14, wherein the change in operating functionality includes a reduction in operating functionality of the system.

16. A system according to claim 14, wherein the change in operating functionality includes a termination of operating functionality of the system.

17. A system according to claim 13, wherein the alarm condition includes a low battery condition.

18. A system according to claim 13, wherein the alarm condition includes a reduced connection with an external portion of the system.

19. A system according to claim 13, wherein the alarm condition includes loss of connection with an external portion of the system.

20. A system according to claim 13, wherein the given alarm condition includes a plurality of different alarm conditions, and wherein there is a different distinctive patient alarm signal for each alarm condition.

21. A system according to claim 13, wherein the patient alarm signal is a vibratory signal.

22. A system according to claim 13, wherein the patient alarm signal is an auditory signal.

23. A system according to claim 13, wherein the patient alarm signal is a visual signal.

24. A system according to claim 13, wherein the patient alarm signal is an electrical stimulation signal.
